(19) United States
(12) Patent Application Publication
(10) Pub. No.: US 2013/0116552 A1
(43) Pub. Date: May 9, 2013

(54) IMPLANTABLE IMAGING ARRANGEMENT AND METHOD FOR USING THE SAME

(71) Applicants: THE GENERAL HOSPITAL CORPORATION, Boston, MA (US); AIR LIQUIDE, Jouy en Josas Cedex (FR)

(72) Inventors: Eman Namati, Belmont, MA (US); William C. Warger, III, Cambridge, MA (US); Guillermo J. Tearney, Cambridge, MA (US); Brett Eugene Bouma, Quincy, MA (US); Carolin Unglert, Boston, MA (US)

(73) Assignees: AIR LIQUIDE, Jouy en Josas Cedex (FR); THE GENERAL HOSPITAL CORPORATION, Boston, MA (US)

(21) Appl. No.: 13/656,426

(22) Filed: Oct. 19, 2012

Related U.S. Application Data
(60) Provisional application No. 61/549,567, filed on Oct. 20, 2011.

(51) Int. Cl. A61B 5/00

(52) U.S. Cl. CPC A61B 5/0077; A61B 5/0066
USPC 600/425; 600/476

(57) ABSTRACT

According to an exemplary embodiment of the present disclosure, apparatus and method can be provided for obtaining information associated with at least one anatomical structure. The exemplary apparatus can include at least one first arrangement which can be configured to provide at least one electro-magnetic radiation to at least one portion of the at least one anatomical structure. The first arrangement(s) can include a configuration which is configured to relatively permanently attach the apparatus to or in the at least one anatomical structure. The exemplary apparatus can further include at least one interferometric arrangement configured to receive a further radiation from the portion(s) which can be associated with the electro-magnetic radiation(s).

### CROSS-REFERENCE TO RELATED APPLICATION(S)

This application is based upon and claims the benefit of priority from U.S. Patent Application Ser. No. 61/549,567 filed Oct. 20, 2011, the entire disclosure of which is incorporated herein by reference.

### FIELD OF THE DISCLOSURE

Exemplary embodiments of the present disclosure relate to implantable and/or attachable devices, and more particularly, to implantable and/or attachable devices that can provide dynamic, functional, and/or progressive imaging and/or treatment.

### BACKGROUND INFORMATION

Analyses have been performed by independent imaging techniques at discrete time points and in controlled settings for determining medical information. While these analyses have been important for the development of diagnostics and treatments for the general public, they are typically approximations for the continuous physiologic and molecular processes that develop in an individual over time. It can be then desirable to create an implantable and/or attachable device that can provide dynamic, functional, and/or progressive imaging and/or treatment to develop personalized treatments and further understanding of disease development.

Accordingly, there may be a need to address at least some of the above-described deficiencies.

### SUMMARY OF EXEMPLARY EMBODIMENTS

According to exemplary embodiments of the present disclosure, an implantable and/or attachable device/apparatus/arrangement can be provided which can perform microscopic structural and functional assessments with optical techniques including, e.g., brightfield, fluorescence, confocal, multi-photon, optical spectroscopy, Raman spectroscopy, coherent anti-Stokes Raman spectroscopy, optical coherence tomography, diffuse optical tomography, full-field quantitative phase, photoacoustic, laser speckle, photodynamic therapy, and/or laser ablation, either individually or in combination.

The exemplary device/arrangement can be miniature and lightweight, and can be implanted and/or attached to an anatomical structure over extended periods of time without hindering the natural motion and function of the anatomical structure.

According to exemplary embodiments of the present disclosure, exemplary devices, apparatus, and arrangements can be provided which can be implanted and/or attached to specimen and can perform microscopic structural and functional assessments. The exemplary apparatus can include at least one first arrangement which can be configured to provide at least one electro-magnetic radiation to at least one portion of the at least one anatomical structure. The first arrangement(s) can include a configuration which is configured to relatively permanently attach the apparatus to or in the at least one anatomical structure. The exemplary apparatus can further include at least one interferometric arrangement configured to receive a further radiation from the portion(s) which can be associated with the electro-magnetic radiation(s). The exemplary apparatus can include a housing that is composed of bio-compatible material or a drug-eluting material thereon and at least partially encloses the first arrangement(s). According to certain exemplary embodiments, the configuration can include at least one of a suction arrangement, a hooking arrangement, an adhesive arrangement or a surface tension arrangement.

According to yet another exemplary embodiment of the present disclosure, the first arrangement(s) can include a beam scanning arrangement, which can include a micro electro-mechanical system (MEMS) or a Risley prism arrangement. The first arrangement(s) can also be further configured to provide an additional radiation which can cause at least one anatomical change to the portion(s).

According to yet another exemplary embodiment of the present disclosure, the interferometric arrangement(s) can be further configured to generate an imaging having optical coherence tomography characteristics.

According to yet another exemplary embodiment of the present disclosure, the exemplary apparatus can further include a drug delivery arrangement which can be configured to provide at least one drug to the portion(s).

These and other objects, features and advantages of the present disclosure will become apparent upon reading the following detailed description of exemplary embodiments of the present disclosure, when taken in conjunction with the appended drawings and claims.

### BRIEF DESCRIPTION OF THE DRAWINGS

Further objects, features and advantages of the present disclosure will become apparent from the following detailed description taken in conjunction with the accompanying drawings showing illustrative embodiments of the present invention, in which:

FIG. 1A is a diagram of an exemplary battery-operated, wireless, implantable arrangement according to an exemplary embodiment of the present disclosure;

FIG. 1B is a diagram of exemplary scanning techniques that can be utilized by the exemplary implantable arrangement according to an exemplary embodiment of the present disclosure;

FIG. 2A is an image of an exemplary attachable probe that can relay electrical and/or optical signals to a processing and recording device according to an exemplary embodiment of the present disclosure;

FIG. 2B is a wire diagram of the exemplary attachable probe shown in FIG. 2A according to an exemplary embodiment of the present disclosure;

FIG. 2C is a diagram of an exemplary implanted probe that relays electrical and optical signals to the processing and recording device that is attached to the outside of the body according to an exemplary embodiment of the present disclosure;

FIG. 3A is a diagram of an exemplary vacuum suction channel within the implantable device for attachment to an anatomic structure according to an exemplary embodiment of the present disclosure;

FIG. 3B is a diagram of an exemplary adhesive and/or surface tension attaching the implantable device to the anatomic structure according to an exemplary embodiment of the present disclosure;

FIG. 3C is a diagram of an exemplary hooking arrangement attaching the implantable device to the anatomic structure according to an exemplary embodiment of the present disclosure;

FIG. 3D is a diagram of an exemplary suturing arrangement attaching the implantable device to the anatomic structure according to an exemplary embodiment of the present disclosure;

FIG. 4 is a diagram of an exemplary implantable probe having a drug-eluting coating to prevent foreign-body reaction and/or provide a means for treatment according to an exemplary embodiment of the present disclosure;

FIG. 5 is a diagram of the exemplary implantable probe that has drug-eluting channels to prevent foreign-body reaction and/or provide an arrangement or a procedure for treatment according to an exemplary embodiment of the present disclosure;

FIG. 6 is a diagram of the exemplary implantable probe that provides an electromagnetic radiation as the activating medium for biomolecular and/or pharmaceutical agents and/or for ablation in order to provide therapy according to an exemplary embodiment of the present disclosure; and

FIG. 7 is an illustration of the exemplary probe attached to excised organs during transfer to assess viability before, during, and/or after transplant according to an exemplary embodiment of the present disclosure.

Throughout the drawings, the same reference numerals and characters, if any and unless otherwise stated, are used to denote like features, elements, components, or portions of the illustrated embodiments. Moreover, while the subject disclosure will now be described in detail with reference to the drawings, it is done so in connection with the illustrative embodiments. It is intended that changes and modifications can be made to the described embodiments without departing from the true scope and spirit of the subject disclosure as defined by the appended claims.

### DETAILED DESCRIPTION OF EXEMPLARY EMBODIMENTS

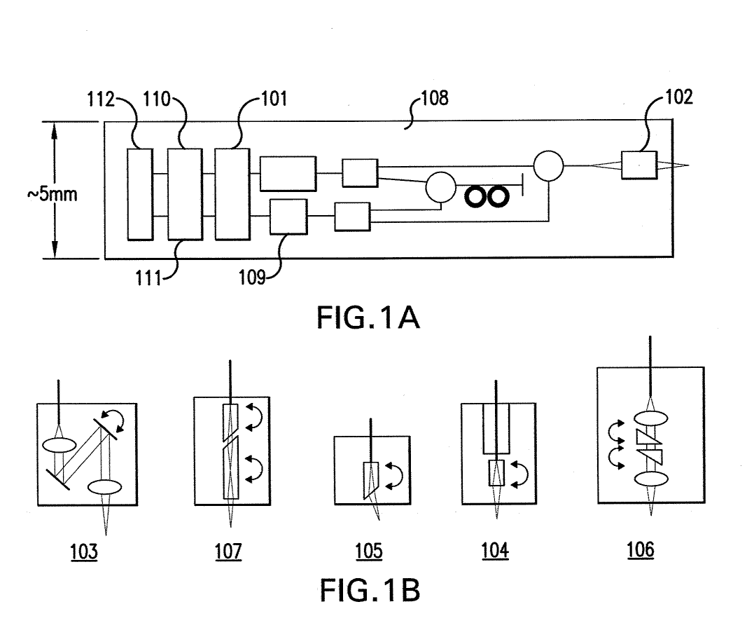

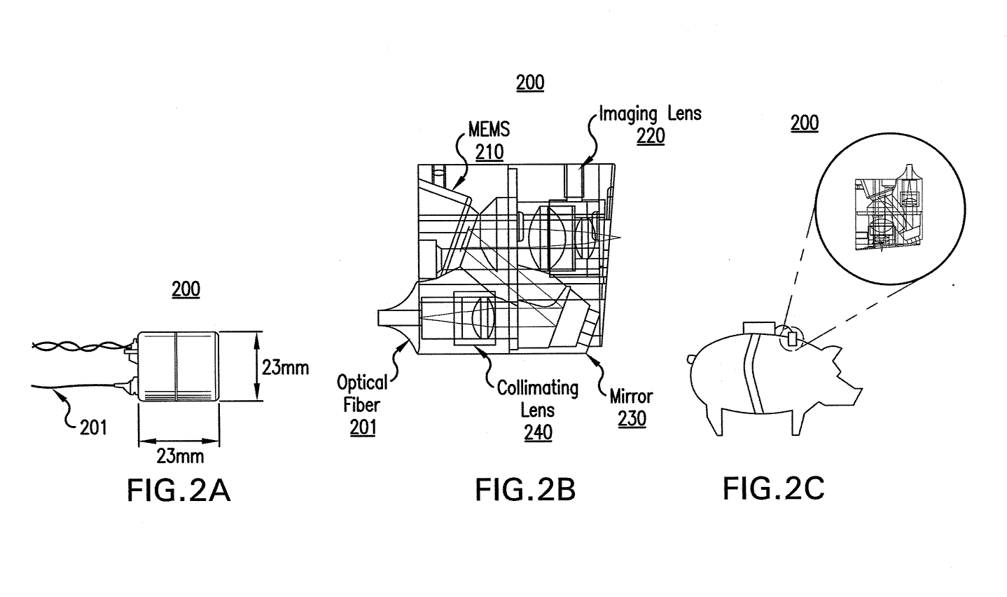

According to certain exemplary embodiments of the present disclosure, an implantable and/or attachable device/arrangement/apparatus can be provided which can facilitate dynamic, functional, and/or progressive imaging and/or treatment. FIG. 1A shows a diagram of an exemplary embodiment of an implantable arrangement 100 according to the present disclosure that can include a light source arrangement 101, such as, e.g., a laser diode, LED, light bulb, or the like, which can illuminate the entire field of view or illuminates individual points within a sample using, e.g., a scanner 102. The exemplary scanner 102 can be or include, for example, any of a micro electromechanical system (MEMS) 103, a resonating fiber 104, an angle-polished rotating fiber 105, single or double rotating prisms 106, a rotating angle-polished gradient index (GRIN) lenses 107, as shown in FIG. 2B, as well as a tuning fork cantilever, a spatial frequency encoding, acousto-optic modulator, or similar. The exemplary apparatus 100 can be configured and/or structured to also direct an electro-magnetic radiation (e.g., light) to the sample and/or a specimen at different wavelengths by use of, for example, a broad-band light source. The electro-magnetic radiation can be guided to the sample using another arrangement 108, such as, e.g., any combination of lenses, optical fiber, deformable minor, mechanical translation, and/or an acousto-optic device.

According to an exemplary embodiment of the present disclosure, the electro-magnetic radiation (e.g., light) returned from the specimen and/or sample can be detected by, for example, a detector 109, such as, e.g., one or more point detectors, one or two dimensional array of detectors, CCD or CMOS camera, or the like. The detected signal can, for example, be recorded on a storage arrangement, such as, e.g., a non-volatile memory 110, RAM, ROM, etc. Further, the detected signal can be transmitted using electrical conductors or a wireless radio frequency transmitter 111, and can be processed by a processing arrangement 110. According to a certain exemplary embodiment of the present disclosure, the exemplary arrangement (e.g., device) 100 can be battery powered. Alternatively, the exemplary arrangement 100 can also be powered remotely via one or more electrical conductors and/or by a radio frequency power transmission 112. According to yet another exemplary embodiment of the present disclosure, the entire exemplary arrangement 100 can be implanted within the body.

According to another exemplary embodiment of the present disclosure, an exemplary attachable probe can be provided. For example, FIGS. 2A and 2B illustrate such exemplary attachable probe 200. As shown in FIGS. 2A and 2B, the exemplary attachable probe 200 can configured and/or structured to be implanted, for example, within a body (e.g., human body, animal body, etc.), and the electrical and/or optical signals can be relayed, e.g., via one or more optical fiber(s) 201 to a processing and/or recording device that can be worn or attached to the outside of the body or can be a standalone instrument, as shown, for example, in a diagram of an exemplary embodiment of FIG. 2C. The exemplary attachable probe 200 can also include a MEMS arrangement 210, an imaging lens arrangement 220, one or more mirrors 230 which transmit the optical signals, and a collimating lens arrangement 240 which is configured to provide the electrical and/or optical signals

The exemplary arrangement 100 and/or the exemplary probe 200 can also be attached to a natural or a man-made optical window to assess anatomical structures where the probe can interfere with the natural physiology or may not be attached.

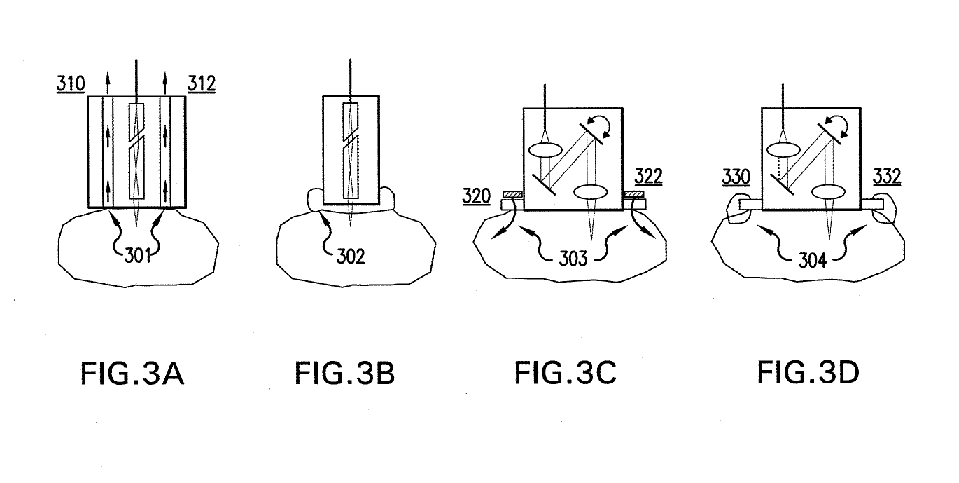

According to yet another exemplary embodiment of the present disclosure, an exemplary device can be provided that can be attached to a site of interest through the application of vacuum suction 301 (e.g., using suction channel(s) 310, 312), an adhesive and/or surface tension 302 with an adhesive, a hooking arrangement 303 (which includes hooks 320, 322), and/or suturing 304 (which includes sutures 330, 332), or the like, as shown, for example, in the diagram of FIGS. 3A-3D.

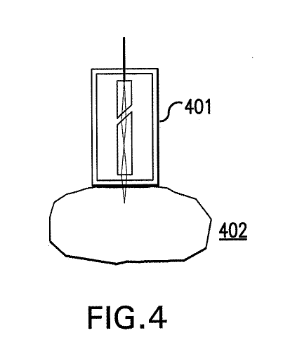

According to still another exemplary embodiment of the present disclosure, as shown in FIG. 4, the exemplary arrangement/device can be housed or coated with a biocompatible material 401 that can be relatively permanently attached upon or within an anatomic structure 402 such that the device can operate while the body undergoes normal and/or strenuous activity over short or extended lengths of time.

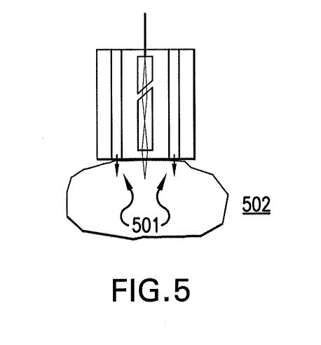

With a further exemplary embodiment of the present disclosure, as shown in FIG. 5, the housing and/or the biocompatible material can be coated and/or contain a drug-eluting or drug-delivering material 501 to prevent foreign-body reaction with respect to the sample 502 and/or facilitate treatment.

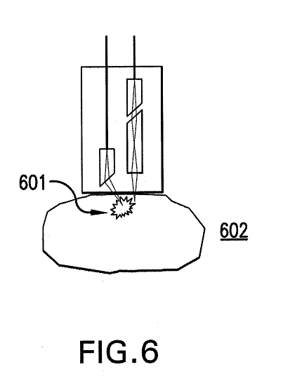

According to a still further another exemplary embodiment of the present disclosure, as shown in FIG. 6, the exemplary arrangement/device can provide an electromagnetic radiation 601 as a primary and/or activating medium for biomolecular and/or pharmaceutical agents or for ablation in order to provide therapy to the sample 602.

Regarding another exemplary embodiment of the present disclosure, a processing apparatus can be provided which can be configured to assess treatment, provide feedback, and/or apply additional therapy with the attachable probe for continual personalized treatment.

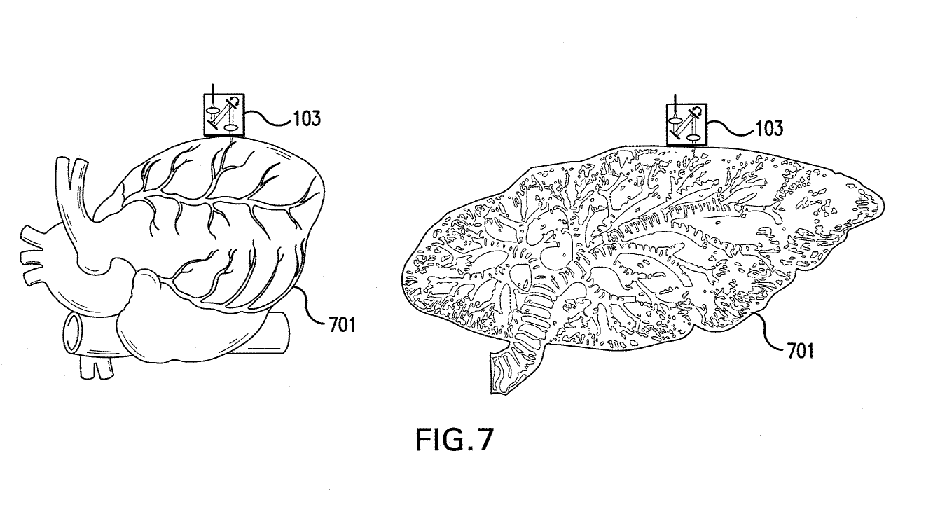

According to yet another exemplary embodiment of the present disclosure, as shown in FIG. 7, the exemplary arrangement/device can be attached to an excised organ 701 during transfer to assess viability before, during, and/or after transplant.

Turning to still yet another exemplary embodiment of the present disclosure, the exemplary arrangement/device can be attached to the peripheral lung in patients to assess Acute Respiratory Distress Syndrome, Ventilator Induced Lung Injury, or Acute Lung Injury.

Further, according to another exemplary embodiment of the present disclosure, the exemplary arrangement/device can be used to assess and detect abnormalities in or near lymph nodes. Pursuant to still another exemplary embodiment of the present disclosure, the exemplary arrangement/device can also evaluate the natural progression of tumor development and the response of chemotherapeutic, photodynamic, laser ablation, and/or natural remedies to tumor interventions.

The exemplary arrangement/device can also be implanted on muscles, joints, tendons, or the like to assess stress, strain, and/or damage during physical activity for orthopedic assessment.

The exemplary arrangement/device can also be implanted and/or attached in order to assess blood oxygenation and the potential for compartment syndrome.

The exemplary arrangement/device can further be implanted and/or attached to assess the progression of internal bleeding.

The foregoing merely illustrates the principles of the present disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication. No. 2005/0018201 on Jan. 27, 2005, U.S. Patent Publication No. 2002/0122246, published on May 9, 2002, U.S. Patent Application No. 61/649,546, U.S. patent application Ser. No. 11/625,135, and U.S. Patent Application No. 61/589,083, the disclosures of which are incorporated by reference herein in their entireties.

It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly incorporated herein in its entirety.

It will thus be appreciated that those skilled in the art will be able to devise numerous systems, arrangements and methods which, although not explicitly shown or described herein, embody the principles of the present disclosure and are thus within the spirit and scope of the present disclosure. Further, various exemplary embodiments described herein can be interchangeably used with all other exemplary described embodiments, as should be understood by those having ordinary skill in the art. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus for obtaining information associated with at least one anatomical structure, comprising:
at least one first arrangement which is configured to provide at least one electro-magnetic radiation to at least one portion of the at least one anatomical structure, wherein the at least one first arrangement includes a configuration which is configured to relatively permanently attach the apparatus to or in the at least one anatomical structure; and
at least one interferometric arrangement configured to receive a further radiation from the at least one portion which is associated with the at least one electro-magnetic radiation.

2. The apparatus according to claim 1, wherein the configuration includes at least one of a suction arrangement, a hooking arrangement, an adhesive arrangement or a surface tension arrangement.

3. The apparatus according to claim 1, wherein the at least one first arrangement includes a beam scanning arrangement.

4. The apparatus according to claim 3, wherein the beam scanning arrangement includes a micro electro-mechanical system (MEMS).

5. The apparatus according to claim 3, wherein the beam scanning arrangement includes a Risley prism arrangement.

6. The apparatus according to claim 1, wherein the at least one interferometric arrangement is further configured to generate an image having optical coherence tomography characteristics.

7. The apparatus according to claim 1, further comprising a housing that at least partially encloses the first arrangement, wherein the housing is composed of bio-compatible material.

8. The apparatus according to claim 1, further comprising a housing that at least partially encloses the first arrangement, wherein the housing includes drug-eluting material thereon.

9. The apparatus according to claim 1, further comprising a drug delivery arrangement which is configured to provide at least one drug to the at least one portion.

10. The apparatus according to claim 1, wherein the at least one first arrangement is further configured to provide an additional radiation which causes at least one anatomical change to the at least one portion.

11. A method for obtaining information associated with at least one anatomical structure, comprising:
providing at least one electro-magnetic radiation to at least one portion of the at least one anatomical structure;
relatively permanently attaching an apparatus performing the method to or in the at least one anatomical structure; and
using at least one interferometric arrangement, receiving a further radiation from the at least one portion which is associated with the at least one electro-magnetic radiation.

12. The method according to claim 11, further comprising using the at least one interferometric arrangement, generating an image having optical coherence tomography characteristics.

13. The method according to claim 11, further comprising, using a drug delivery arrangement, providing at least one drug to the at least one portion.

14. The method according to claim 11, further comprising providing an additional radiation which causes at least one anatomical change to the at least one portion.
